(19) United States
(12) Patent Application Publication
(10) Pub. No.: US 2023/0197270 A1
(43) Pub. Date: Jun. 22, 2023

(54) DETERMINING A HEALTH RISK

(71) Applicant: Micron Technology, Inc., Boise, ID (US)

(72) Inventors: Yixin Yan, Boise, ID (US); Libo Wang, Boise, ID (US); Ramya B Tatapudi, Boise, ID (US)

(21) Appl. No.: 17/858,938

(22) Filed: Jul. 6, 2022

Related U.S. Application Data
(60) Provisional application No. 63/291,564, filed on Dec. 20, 2021.

(51) Int. Cl. G16H 50/20; G16H 40/67

(52) U.S. Cl. CPC G16H 50/20; G16H 40/67; G16H 10/60

(57) ABSTRACT

Methods, apparatuses, and non-transitory machine-readable media associated with a health risk determination are described. A health risk determination can include receiving first signaling from a first source configured to monitor behavior of a patient and receiving second signaling from a second source configured to monitor environmental data associated with the patient. The health risk determination can include writing data based at least in part on the first signaling and the second signaling and determining a health risk for the patient based on the first signaling and the second signaling. The health risk determination can include identifying output data representative of a health risk response plan for the patient based at least in part on input data representative of the health risk and additional patient data stored in the memory resource or other storage and transmitting the output data representative of the health risk response plan via third signaling.

### PRIORITY INFORMATION

This application claims the benefit of U.S. Provisional Application No. 63/291,564, filed Dec. 20, 2021, the contents of which are incorporated herein by reference.

### TECHNICAL FIELD

The present disclosure relates generally to apparatuses, non-transitory machine-readable media, and methods associated with determining a health risk, including the use of machine learning.

### BACKGROUND

Memory resources are typically provided as internal, semiconductor, integrated circuits in computers or other electronic systems. There are many different types of memory, including volatile and non-volatile memory. Volatile memory can require power to maintain its data (e.g., host data, error data, etc.). Volatile memory can include random access memory (RAM), dynamic random-access memory (DRAM), static random-access memory (SRAM), synchronous dynamic random-access memory (SDRAM), and thyristor random access memory (TRAM), among other types. Non-volatile memory can provide persistent data by retaining stored data when not powered. Non-volatile memory can include NAND flash memory, NOR flash memory, and resistance variable memory, such as phase change random access memory (PCRAM) and resistive random-access memory (RRAM), ferroelectric random-access memory (FeRAM), and magnetoresistive random access memory (MRAM), such as spin torque transfer random access memory (STT RAM), among other types.

Electronic systems often include a number of processing resources (e.g., one or more processing resources), which may retrieve instructions from a suitable location and execute the instructions and/or store results of the executed instructions to a suitable location (e.g., the memory resources). A processing resource can include a number of functional units such as arithmetic logic unit (ALU) circuitry, floating point unit (FPU) circuitry, and a combinatorial logic block, for example, which can be used to execute instructions by performing logical operations such as AND, OR, NOT, NAND, NOR, and XOR, and invert (e.g., NOT) logical operations on data (e.g., one or more operands). For example, functional unit circuitry may be used to perform arithmetic operations such as addition, subtraction, multiplication, and division on operands via a number of operations.

Artificial intelligence (AI) can be used in conjunction memory resources. AI can include a controller, computing device, or other system to perform a task that normally requires human intelligence. AI can include the use of one or more machine learning models. As described herein, the term “machine learning” refers to a process by which a computing device is able to improve its own performance through iterations by continuously incorporating new data into an existing statistical model. Machine learning can facilitate automatic learning for computing devices without human intervention or assistance and adjust actions accordingly.

### BRIEF DESCRIPTION OF THE DRAWINGS

FIG. 1 is a functional diagram representing a system for health risk determination in accordance with a number of embodiments of the present disclosure.

FIG. 2A is another functional diagram representing a system for health risk determination in accordance with a number of embodiments of the present disclosure.

FIG. 2B includes health risk trend charts in accordance with a number of embodiments of the present disclosure.

FIG. 3 is another functional diagram representing a processing resource in communication with a memory resource having instructions written thereon in accordance with a number of embodiments of the present disclosure.

FIG. 4 is yet another functional diagram representing a processing resource in communication with a memory resource having instructions written thereon in accordance with a number of embodiments of the present disclosure.

FIG. 5 is a flow diagram representing an example method for health risk determination in accordance with a number of embodiments of the present disclosure.

### DETAILED DESCRIPTION

Systems, devices, and methods related to a health risk determination are described. A health risk can include a chance or likelihood that something with harm or otherwise affect a patient's health. This can include disease, allergies, mental health conditions, developmental delays, etc. A developmental delay includes a condition of a child or adult being less developed mentally or physically than is normal for the child's age. Children and adults are unique and have their own developmental progress and milestones. Parents and caregivers may seek to identify health risks including developmental delays early but may be limited by limited doctor visits and limited real time evidence. In such instances, a health care provider may focus on the child's behaviors, but may not consider hereditary factors, environmental influences, or social interactions, among others.

Examples of the present disclosure can allow for customized, individual early health risk detection to determine the health risk and a health risk response plan to potentially prevent harm or adverse health effects including, for instance, developmental delays. Early detection can allow for timely intervention and improved treatment and outcomes. Examples can include the use of a machine learning model or models utilizing real-time and ad-hoc patient health data, information from the medical field, and environmental data to determine a health risk and a health risk response plan.

Examples of the present disclosure can include a method for determining a health risk including receiving at a first processing resource, first signaling from a first source configured to monitor behavior of a patient and receiving at the first processing resource, second signaling from a second source configured to monitor environmental data associated with the patient. The method can include writing from the first processing resource to a memory resource coupled to the first processing resource data that is based at least in part on a combination of the first signaling and the second signaling and determining, at the first processing resource or a different, second processing resource, a health risk for the patient based on the first signaling and the second signaling.

Examples can include identifying, at the first processing resource or the different, second processing resource, output data representative of a health risk response plan for the patient based at least in part on input data representative of the health risk and additional patient data stored in a portion of the memory resource or other storage accessible by the first processing resource and transmitting the output data representative of the health risk response plan via third signaling.

Signaling can be received from a radio or radios in communication with a processing resource or processing resource configured to perform particular tasks (e.g., monitor patient health data of a patient). As used herein, the use of a radio can include the transmission and/or reception of information through intervening media (e.g., air, space, nonconducting materials, etc.). This can include, for instance, radio waves or other wireless communication and/or signaling including but not limited to cellular communication, one-way communication, two-way communication, radar, radiolocation, radio remote control, satellite communication, Wi-Fi, 3G, 4G, 5G, and/or other communication standards, among others. In some examples, the use of a radio can include wired transmission and/or reception of information.

In the following detailed description of the present disclosure, reference is made to the accompanying drawings that form a part hereof, and in which is shown by way of illustration how one or more embodiments of the disclosure can be practiced. These embodiments are described in sufficient detail to enable those of ordinary skill in the art to practice the embodiments of this disclosure, and it is to be understood that other embodiments can be utilized and that process, electrical, and structural changes can be made without departing from the scope of the present disclosure.

It is also to be understood that the terminology used herein is for the purpose of describing particular embodiments only and is not intended to be limiting. As used herein, the singular forms “a,” “an,” and “the” can include both singular and plural referents, unless the context clearly dictates otherwise. In addition, “a number of,” “at least one,” and “one or more” (e.g., a number of memory devices) can refer to one or more memory devices, whereas a “plurality of” is intended to refer to more than one of such things. Furthermore, the words “can” and “may” are used throughout this application in a permissive sense (i.e., having the potential to, being able to), not in a mandatory sense (i.e., must). The term “include,” and derivations thereof, means “including, but not limited to.” The terms “coupled,” and “coupling” mean to be directly or indirectly connected physically or for access to and movement (transmission) of commands and/or data, as appropriate to the context.

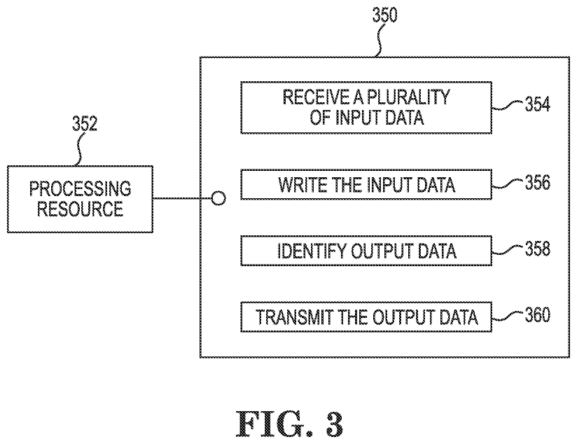

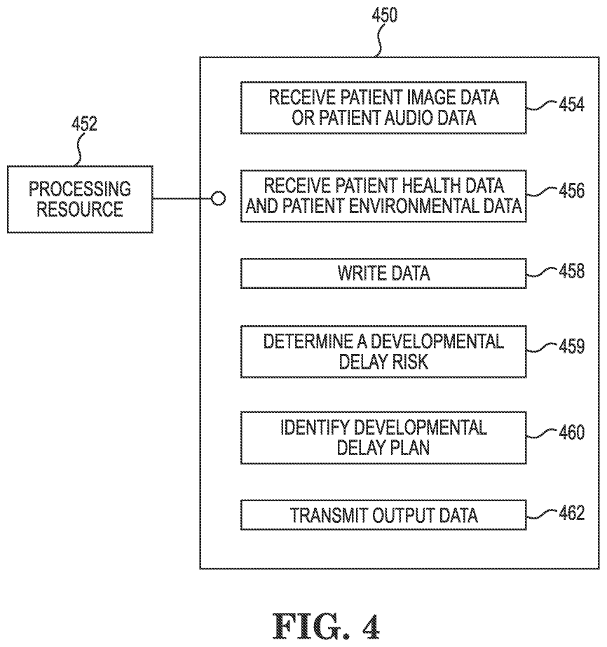

The figures herein follow a numbering convention in which the first digit or digits correspond to the figure number and the remaining digits identify an element or component in the figure. Similar elements or components between different figures can be identified by the use of similar digits. For example, 350 can reference element “50” in FIG. 3, and a similar element can be referenced as 450 in FIG. 4. As will be appreciated, elements shown in the various embodiments herein can be added, exchanged, and/or eliminated so as to provide a number of additional embodiments of the present disclosure. In addition, the proportion and/or the relative scale of the elements provided in the figures are intended to illustrate certain embodiments of the present disclosure and should not be taken in a limiting sense.

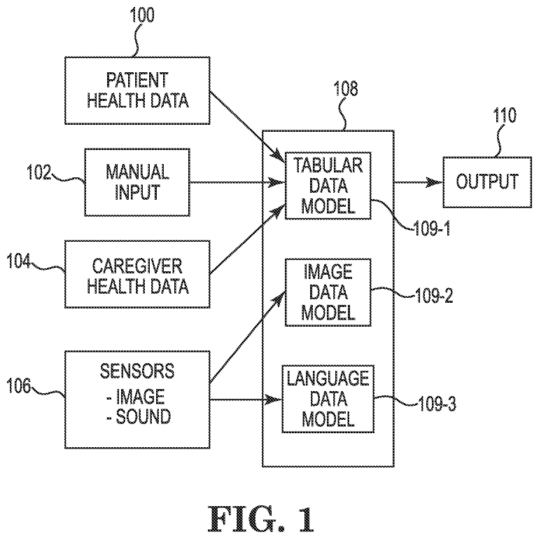

FIG. 1 is a functional diagram representing a system for health risk determination in accordance with a number of embodiments of the present disclosure. The system can include a health risk tool 108 that uses a machine learning model or models to determine a health risk, health risk response plan, or both, for a patient. The health risk can include a likelihood at a particular period or point in time that the patient is at risk, for instance of a developmental delay, and the health risk response plan can include a plan of action, both immediate and long-term to reduce the health risk.

The health risk tool 108 can include, for instance, a tabular data machine learning model 109-1 (e.g., tree-based machine learning model) that performs multi-class classification on tabular data (e.g. numerical data, categorical data, etc.), an image data machine learning model 109-2 (e.g., a convolution neural network machine learning model) to perform multi-class classification on image data (e.g., videos, images, etc.), a language data model 109-3 (e.g., speech recognition machine learning model) to perform multi-class classification on audio data (e.g., sound data, language data, etc.) or any combination thereof. Other machine learning models may be part of the health risk tool 108. The health risk tool 108 can include, in some examples, a processing resource in communication with a memory resource that utilizes AI (including the machine learning models 109-1, 109-2, 109-3) to determine a health risk, a health risk response plan, or both. Put another way, the health risk tool 108 and associated AI determines a health risk (e.g., developmental delay risk) and/or creates a plan of action for a patient based on data available to the health risk tool 108 including, but not limited to, patient health data and environmental data.

The health risk tool 108 and associated AI (e.g., including machine learning models 109-1, 109-2, 109-3 can be trained using a training dataset or training datasets. For instance, a training dataset can include a set of examples used to fit parameters of the AI. For instance, the training dataset can include data associated with patient health data, generic health risk data (e.g., common developmental delays and treatments), and environmental data, among others for each machine learning model 109-1, 109-2, 109-3. In some examples, the health risk tool 108 and associated machine learning models 109-1, 109-2, 109-3 can also be trained using new input data (e.g., new data from patients, providers, environmental sensors, research data, etc., among others). In some examples, the health risk tool 108 and associated trained machine learning models 109-1, 109-2, 109-3 can include continuous learning of the machine learning models 109-1, 109-2, 109-3 and re-calibration of the machine learning models 109-1, 109-2, 109-3.

The health risk tool 108 can receive input data from a plurality of sources. The input data can be encrypted, in some examples. Sources can include a database of generic patient and/or treatment information (e.g., relating to developmental delays, allergies, other health risks, etc.), patient health information sources (e.g., personal tracking devices, personal medical devices, insurance information, cameras, audio collection devices, mobile applications, manual input, text/language tools, patient health data, etc.), providers (e.g., health care provider data), and environmental information sources (weather sensors, cameras, motion detectors, light detectors, diet sensors, audio collection devices, etc.). For instance, the database of generic patient and/or treatment information may include common symptoms, visuals, treatments, and other data associated with particular health conditions (e.g., allergies, developmental delays, mental health conditions, diseases, etc.).

Patient health data can be received from personal tracking devices such as a global positioning service (GPS) on a mobile device including a patient location, a sleep tracking application, dietary tracking application, fitness tracking application, or other personal tracking device, among others. Patient health data can be received from personal medical devices in real time (e.g., heartrate monitor, emotion monitor (e.g., based on vital signs), sleep monitor, body temperature, oxygen level monitors, etc.). Patient health data can also be collected using cameras, audio collection devices, and other devices that can monitor in real time patient actions and speech. For instance, a camera may collect a child's attempts to stand, attempts to crawl, attempts to roll, walking patterns, etc., while an audio collection device captures a child's apparent stutter and/or social interactions. In some instances, the camera and audio collection device are the same device (e.g., in-home monitoring systems).

In some examples, patient health data can be received ad-hoc, for instance as data from a health care provider (e.g., blood test results, vital signs, physician notes, age, medical history, genetic testing results, etc.) or a patient can manually input patient data such as address or birthday information and/or patient health data such as current symptoms, current ailments, medication tracking, family health history, allergies, patient health history, etc. via an application on a computing device and associated with the health risk tool 108.

For instance, at 100, individual patient data can be monitored and collected. For example, patient health data can be monitored, using a health sensor, health monitor, wearable device, mobile device, etc. of the patient. This can also include genetic information gathered using an at-home toolkit or other method. A patient's heartrate, blood pressure, body temperature, oxygen levels, sleep patterns, motions (e.g., standing/sitting), diet, exercise patterns, exercise levels, and other data may be monitored. In some examples, health care provider data can be received including, for instance, vital signs from previous visits, test results (e.g., genetic testing), age, and medical history, among others.

At 102, manual input can be received. This can include any of the aforementioned patient health data, but may be provided in a manual matter, for instance, via a mobile questionnaire requesting age, gender, height, weight, etc. Manual input can also include environmental data including social and economic status, family size and makeup, circle of friends and friends' mannerisms, patient's school, caregiver workplaces, work hours, etc. Manual input may also include uploaded historical photographs and videos (e.g., child's first steps, siblings walking, speeches, etc.).

At 104, caregiver health data can be monitored and collected. This can include input collected from a health sensor, health monitor, wearable device, mobile device, etc. of the caregiver, medical records from a health provider, and/or genetic information. In some examples, behavioral data associated with the caregiver may be collected using cameras, audio devices, etc.

At 106, data from sensors, including image sensors (e.g., cameras or other sensors) and sound sensors (e.g., audio collection devices) can be collected and monitored. Data from sensors can include aforementioned patient and caregiver health data collected using aforementioned personal sensors and/or cameras, audio collection devices, etc. For instance, a home monitoring camera may collect real time video of a child attempting to walk. The video may be separated into image sequences (e.g., image frames). The audio collection devices may collect and provide speech patterns, yelling, or other environmental data. In some examples, additional environmental data (e.g., humidity, lighting, temperature, etc.) can be collected at sensors and received at the health risk tool 108 as part of the individual patient data, caregiver health data, manual input, or received via sensors.

The health risk tool 108 can consider the data received to determine potential health risk factors for the patient. For instance, the health risk tool 108 includes the tabular machine learning model 109-1 that uses tabular data to perform multi-class classification, the image data machine learning model 109-2 (e.g., a convolution neural network model) to perform multi-class classification, and the language data model 109-3 (e.g., a speech recognition model) to perform multi-class classification, among other models. The models 109-1, 109-2, 109-3 can be used to determine and output at 110 how much and what data is needed to determine a health risk, and/or suggestions for patients or caregivers including strategies or suggestions to consult a physician.

For instance, in a non-limiting example, input data may indicate the patient has similar environmental factors and/or health conditions to other patients in medical research data (e.g., socioeconomic status, speech development patterns, genetic patterns, etc.) who experience developmental delays. The health risk tool 108 and associated models 109-1, 109-2, 109-3 can receive input data representing patient health and environmental factors and monitor the input data and potential health risk (e.g., potential developmental delay risk). Other health risk factors may be monitored and considered. Output data, at 110, can also include warning transmissions to a computing device of a patient, healthcare provider, or caregiver to alert the patient or caregiver of a health risk (e.g., potential stutter, potential allergy, etc.) and provide a health risk response plan. In some examples, the output data, at 110, can include an alert sent to a non-mobile device such as a television screen, personal computer, refrigerator display, or smart device (e.g., smart speaker), among others.

In some examples, different sources and associated data may be assigned different weights within the health risk tool 101. For instance, a source determined to provide data more likely to predict a health risk may be given more weight than a source determined to provide data less likely to predict a health risk. In some instance, inputs can carry the same weights. For example, patient health data, manual input data and caregiver health data can be received at 100, 202, and 104 by the tabular data model 109-1. Image data and sound data (e.g., audio data, language data, etc.) can be received at 106 by the image data model 109-2 and the language data model 109-3, respectively. Each model 109-1, 109-2, 109-3 can classify the data received and provide an output in the form of a likelihood of a health risk (e.g., likelihood of a developmental delay).

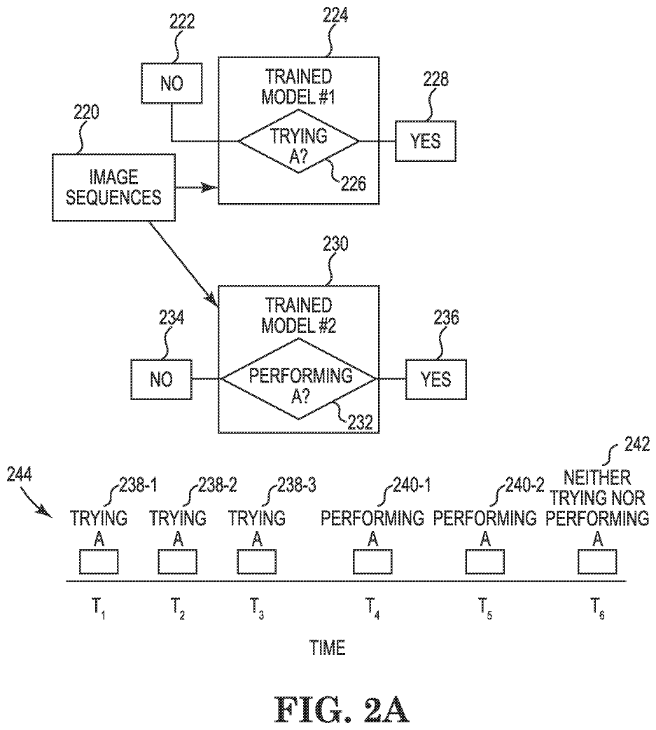

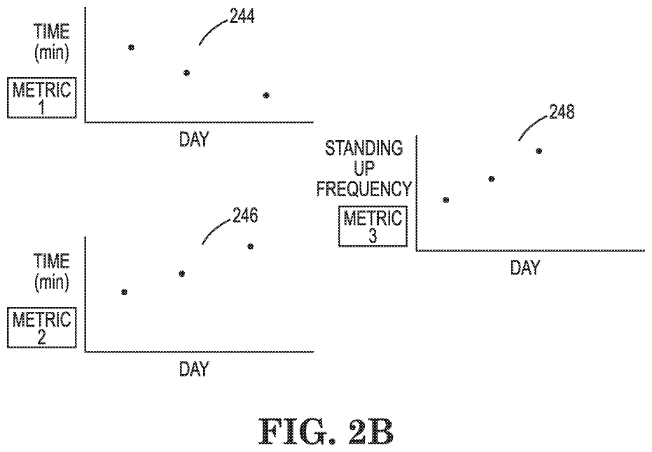

In some examples, the tabular data model 109-1 can provide a baseline for the health risk determination (e.g., a health risk probability). The image data model 109-2 can be used to determine trends in a patient's behavior to increase the accuracy of the health risk determination. For instance, as will be discussed further herein with respect to FIGS. 2A and 2B, health risk predictors can be plotted based on collected images, and data received at the image data model 109-2 can be weighted to increase the accuracy of the health risk determination.

Data input into the models 109-1, 109-2, 109-3 can be weighted, as can the outputs of the models 109-1, 109-2, 109-3. For instance, patient and caregiver health data may be given a higher weight, whereas manually input relationship status data may be give a lower weight. The outputs of the models 109-1, 109-2, 109-3 can be considered and an overall health risk can be output at 110. For instance, the health risk may range from 0 to 1, with 1 being a highest risk, and 0 being a lowest risk.

The health risk and the health risk response plan, as will be discussed further herein, can be used to assist a patient to reduce a risk of experiencing a health condition, prevent a health condition, or both. For instance, a patient may be advised to seek medical guidance if a risk is at or above a particular threshold (e.g., 0.7) or provided with suggestions to proceed if the risk is below the particular threshold.

FIG. 2A is another functional diagram representing a system for health risk determination in accordance with a number of embodiments of the present disclosure. The system can include a health risk tool (e.g., including models 224 and 230) that may be analogous to health risk tool 108, and/or the devices described with respect to FIGS. 3-4 including processing resources 352, 452 and memory devices 350, 450. In the example illustrated in FIG. 2A, the models 224 and 230 are image data machine learning models, and a patient is attempting to perform a task, for instance, a child is attempting to stand up.

FIG. 2B includes health risk trend charts 244, 246, 248 in accordance with a number of embodiments of the present disclosure. For ease of discussion FIGS. 2A and 2B will be described together herein. Each health risk trend chart 244, 246, 248 is associated with a different metric (e.g., Metric 1, Metric 2, Metric 3) associated with a health risk.

At 220, image sequences are gathered. For instance, a home monitoring system (e.g., security system) captures images and videos of a child. Video clips may be cut into image sequences such that each image is a frame. For each image, the health risk tool may identify and/or classify the image as the child trying to stand up (“Trying A” 226) with a “yes” classification 228 or a “no” classification 222. The trained model #1 224 can be trained using historical data such as historical images labeled with “trying to stand up” or “not trying to stand up” and a model such as a convolution neural network model, among others.

Using trained model #2 230, the health risk tool may identify and/or classify the image as the child standing up (“Performing A” 232) with a “yes” classification 236 or a “no” classification 234. The trained model #2 230 can be trained using historical data such as historical images labeled with “standing up” or “not standing up” and a model, such as a convolution neural network model, among others.

A plurality of images and image sequences can be analyzed using the health risk tool and associated models 224, 230, and a timeline 244 of results can be produced. For instance, attempts to stand 238-1, 238-2, 238-3, successful stands 240-1, 240-2, and instances of neither attempting to stand nor standing 242 can be tracked. A time difference between a first image of standing up 240-1 and a first image of trying to stand up 238-1 can be determined by the health risk tool, and this can be compared to a trend chart 244 with similar children (e.g., based on age, gender, race, geography, etc.). For instance, the trend chart 244 illustrates such a metric (e.g., Metric 1) and illustrates a shorter time period over a number of days between first attempting to stand and actually standing. Similar, Metric 2 can include a time difference between a first standing up 240-1 and a last standing up 240-2 and can be determined by the health risk tool and compared to a trend chart 246 of similar aged children. In some examples, a frequency of trying to stand up can be determined and compared with similar children (e.g., based on age, gender, race, geography, etc.), for instance as Metric 3 as illustrated in trend chart 248. Machine learning models of the health risk tool can use these metrics to output data representative of a health risk (e.g., risk of developmental delay) or health risk response plan (e.g., practice plan, consult physician, etc.). For example, trends of time lengths increasing between starting and ending standing 240-1, 240-2 may indicate improvement in muscle strength of a child and reduced developmental delay risk. Put another way, a child's development matching the trend charts 244, 246, 248 may indicate no developmental delay, while a deviation may indicate a potential developmental delay. The metrics and matching and/or deviations therefrom may be considered in the determination, as well. While three metrics are illustrated herein, more or fewer metrics may be determined and utilized in a health risk determination.

While Trying A 226 and Performing A 232 are described herein with respect to a child standing, other developmental tasks or health measures such as other gross motor skills and fine motor skills may be analyzed using the health risk tool. In some examples, the classifications of Trying A 226 and Performing A 232 can be cleared and re-collected once there is a switch from Performing A 232 to Trying A 226. In some instances, a health risk tool can track regression (e.g., time increases between successful standing) and provide health risk response recommendations (e.g., “consult physician for apparent regression”).

For instance, video and voice recordings of parent-child conversations can be collected, and a trained model can be used to determine if the parent is yelling at the child (e.g., “yes” or “no”). In the same non-limiting example, video and voice recordings of parent-child interactions can be collected, and a trained model can be used to determine if the parent is reading books to the child (e.g., “yes” or “no”). A wearable device and associated application may record and analyze whether the child is talking a lot with friends or remaining more silent in a social circle. In such an example, a trained model can be used to determine if the child is more silent (e.g., “yes” or “no”). This gathered data can be used to determine if the child is at risk for a developmental delay based on these environmental factors, their time of occurrence, and/or other data (e.g., patient health data, genetic data, etc.). For instance, positive and negative influences, as well as patterns may be used to identify triggers or warning signs of developmental delays, mental health conditions, or other health risks. For example, in the previous example, a child that stutters only when yelled at by a caregiver may not have a stuttering condition, but rather is nervous in certain situations. This may be addressed with changes in environmental conditions.

FIG. 3 is another functional diagram representing a processing resource 352 in communication with a memory resource 350 having instructions 354, 356, 358, 360 written thereon in accordance with a number of embodiments of the present disclosure. The device illustrated in FIG. 3 can be a server or a computing device (among others) and can include the processing resource 352. The device can further include the memory resource 350 (e.g., a non-transitory MRM), on which may be stored instructions, such as instructions 354, 356, 358, 360. The device, in some examples, may be analogous to health risk tool 108 and/or the device described with respect to FIG. 4 including processing resources 452 and memory resource 450. Although the following descriptions refer to a processing resource and a memory resource, the descriptions may also apply to a system with multiple processing resources and multiple memory resources. In such examples, the instructions may be distributed (e.g., stored) across multiple memory resources and the instructions may be distributed (e.g., executed by) across multiple processing resources.

The memory resource 350 may be electronic, magnetic, optical, or other physical storage device that stores executable instructions. Thus, the memory resource 350 may be, for example, non-volatile or volatile memory. In some examples, the memory resource 350 is a non-transitory MRM comprising RAM, an Electrically-Erasable Programmable ROM (EEPROM), a storage drive, an optical disc, and the like. The memory resource 350 may be disposed within a controller and/or computing device. In this example, the executable 354, 356, 358, 360 can be “installed” on the device. Additionally, and/or alternatively, the memory resource 350 can be a portable, external or remote storage medium, for example, that allows the system to download the instructions 354, 356, 358, 360 from the portable/external/remote storage medium. In this situation, the executable instructions may be part of an “installation package”. As described herein, the memory resource 350 can be encoded with executable instructions for health risk determination.

The instructions 354, when executed by a processing resource such as the processing resource 352 can include instructions to receive at the processing resource 352, the memory resource 350, or both, a plurality of input data from a plurality of sources, the plurality of sources comprising at least two of: a mobile device of a patient, a medical device, a portion of the memory resource or other storage, manually received input, and environmental sensors (e.g., cameras, temperature sensors, etc.). In some examples, the plurality of sources can include computing device data, application data (e.g., diet monitoring application, fitness application, etc.), which may be stored on the mobile device, the memory resource 350, the other storage, or a combination thereof. The plurality of input data, for instance, can include patient health data, generic health risk data, environmental data, or any combination thereof.

For example, the processing resource 352, the memory resource 350, or both, can receive health data specific to the patient (e.g., heartrate, blood pressure, genetic markers, speech patterns, gross motor skill performance, fine motor skill performance, weight, etc.) in an ad-hoc or real time manner as patient health data. The individual patient data, which may be referred to as patient health data, can include health data specific to the patient, and can be received from the plurality of sources including, for instance, sensors, wearable devices or other smart devices (e.g., smartphones), and medical examinations. The sensors can include devices that detect or measure a physical property and record, indicate, or otherwise report it. An example sensor is an electrocardiogram (ECG). In some examples, sensor include cameras and audio collection devices (e.g., home monitoring systems).

The wearable devices or other smart device can include sensors, in some examples, and can use those sensors to gather data including body temperatures, oxygen levels, sleep patterns, changes in motion or other motion (e.g., sitting to standing), among others. The medical examination information can include data collected at a healthcare provider's (e.g., doctor) office or exam such as vital signs, weight etc.

The processing resource 352, the memory resource 350, or both can receive medical research data, publication data, big data, etc. associated with developmental delays. This data, for instance, may come from generic databases of developmental data (e.g., common symptoms, factors among age groups, etc.), among other sources. For instance, information associated with the medical field can be received. This can include, for instance, receiving up-to-date literatures on particular health risk preventions and treatments (e.g., developmental delays, allergies, other diseases, etc.) or gathering data from a hospital or other database for different types of patients, such as infants, children, adults, seniors, patients with particular conditions, etc.

In some examples, the processing resource 352, the memory resource 350, or both, can receive environmental data from sensors or other sources including, for instance, temperature, dietary information/eating habits, screen time information, humidity information, light information, etc. This data can be gathered using sensors including, for instance, sensors around the home (e.g., cameras, motion detectors, etc.), temperature sensors, screen time monitors, diet monitors, etc.

Environmental factors can influence health, including development. The development can be affected by both hereditary and environmental factors associated with biological families and/or other caregivers. Environmental factors such as whether a child lives with one parent, both parents, other relatives, or another caregiver can influence development, as well as caregiver income, education, ethnic background, cultural background, a child's nutrition, and diet structure of the family, among others. Caregiver-child interactions such as spoken languages, attachment, physical interactions, emotional interactions, etc. can be influential, as well.

Community and peer interactions can also influence child development. Education levels of peers and conversations had with peers may affect language development, social and active interactions with peers may affect development, and interactions at school and other community settings can influence a child's development.

In such examples, cameras may capture images and videos of details of caregiver-child interactions or interactions with peers or others. Phones and wearable devices may record conversations between a child and a caregiver, peer, teacher, etc., books read to a child, and/or daily meals and snacks. A child's emotions or other mental health indicators may be monitored throughout the day with wearable devices, for instance using heartrate, breathing rate, etc. This environmental data can be received at input data, for instance at 354.

In some instances, risks and triggers in the environment and how they may impact the patient's personal health can be identified using patient health data, health care provider data, and environmental data. For instance, environmental risks and/or triggers may include caregiver interactions, quick situation changes, screen time, certain foods, medications, and light intensity, among others. Based on historic and current patient data, historic and current environmental data, and historic and current health care provider data, the machine learning models can determine when environmental factors have affected the patient, to what extent the environment factors affected the patient, whether the environmental factors occurred within a threshold time of a flagged action or event (e.g., stutter, allergic reaction, etc.), and how different areas of the patient's health or development were affected by the environmental factors.

The instructions 356, when executed by a processing resource such as the processing resource 352 can include instructions to write from the processing resource 352 to the memory resource 350 the received plurality of input data, and the instructions 358, when executed by a processing resource such as the processing resource 352 can include instructions to identify, using a plurality of machine learning models, at the processing resource 352 or a different processing resource output data representative of a developmental delay plan including a proposed action to identify the developmental delay, address the developmental delay, or both, based at least in part on input data representative of the data written from the processing resource 352. The plurality of machine learning models can include a speech recognition model to perform multi-class classification (e.g., using audio or language data), a convolutional neural network model to perform multi-class classification (e.g., using image data), and a machine learning model using tabular data to perform multi-class classification, among others.

In some examples, identifying the output data representative of the developmental delay plan can be based at least in part on generic developmental delay treatment information, patient information, generic child or adult development information, patient medical history information, or any combination thereof stored in a portion of the memory resource 350 or other storage (e.g., additional memory resource, cloud storage, etc.) accessible by the processing resource 352. Put another way, the instructions 358 can be executable to identify at the processing resource 352 or a different processing resource output data representative of the developmental delay plan using the machine learning models (e.g., trained machine learning models), and the memory resource 350 or other storage can include databases of information accessible by the processing resource 352 for use in the machine learning models. In some examples, the database information may be used to train the machine learning models.

In some examples, different input data can be used by a health risk tool to make different determinations. For instance, an individual developmental baseline can be established using patient health data and a tabular machine learning model. The developmental baseline may establish where the child is developmentally (e.g., walking, talking, fine motor skills, etc.) The health risk tool can utilize the tabular machine learning model that considers the patient health data to establish a developmental baseline including an estimated threshold for the patient at which a developmental delay becomes more likely (e.g., not standing by a certain age). Another machine learning model can analyze image data (e.g., video frames) and in one example, can consider when a patient's first attempt at standing occurred, as well as any attempts and/or successes. The machine learning models can use this information, along with any historic patient health data, newly received ad-hoc patient health data, audio data (e.g., language data associated with speech, environmental conditions, etc.) and real time received patient health data to establish and update the developmental baseline. In some examples, the developmental baseline may be associated with a different health condition, such as a skin allergy. The developmental baseline may be a condition of the skin before exposure to an allergen, for instance.

Deviations from the developmental baseline can be monitored and flagged using patient health data and environmental data. For instance, as real time data and ad hoc data is received, whether patient health data or environmental data, the machine learning models can monitor it and determine if it deviates from the developmental baseline. For instance, if input data associated with a patient's speech patterns are received at the processing resource 352, memory resource 350, or both, and a machine learning model (e.g., a speech recognition machine learning model) indicates it deviates from the developmental baseline (e.g., stutter developing), the speech pattern and time can be flagged. Similar, if input data representative of a child being yelled at received at the processing resource 352, memory resource 350, or both, is determined by a machine learning model (e.g., a model collecting language or image data) to be too high, the situation can be flagged. In some examples, one or more deviations may result in a change to the developmental baseline, a transmitted health risk warning, or both.

In some examples, risk factors associated with the patient can be identified using patient health data, health care provider data, and environmental data. For instance, input data representative of patient health data may indicate a patient has a genetic marker for a particular disease. One of the machine learning models may detect this as a risk factor of the patient and adjust a weight factor of genetics when determining a developmental baseline, request more frequent monitoring, or both. Similar, one of the machine learning models may consider information from a health journal indicating that a particular amount of screen time may affect development. The machine learning model may detect this as a risk factor of the patient, consider historic screen time data (e.g., received as environmental data), adjust a weight factor of screen time when determining a developmental baseline, and request more frequent monitoring, or any combination thereof.

In some examples, health care provider data such as big data from the medical field can be classified and deciphered. For instance, a tabular machine learning model may consider data received from health journals, press articles, medical research, etc., and determine how the data applies to the patient. For instance, the tabular machine learning model may disregard or assign a low weight to input representative of medical research performed only on elderly men when the patient is a young girl.

The instructions 360, when executed by a processing resource such as the processing resource 352 can include instructions to transmit the output data representative of the developmental delay treatment plan to the patient, a health care provider, a caregiver, or any combination thereof. For instance, the patient, the health care provider, or the caregiver can receive a notification of a developmental delay risk and/or an action to take to reduce the risk. A level of risk may be provided (e.g., high, medium, low, etc.), in some examples. For instance, early and personalized ideas to prevent a developmental delay or other health risk can be provided. The ideas can be determined using the established development baseline, baseline deviations, patient risk factors, classified and deciphered healthcare provider data, and environmental risks and triggers. For instance, a patient may be instructed to avoid interactions with a stressful environmental situation to prevent a stutter or avoid a particular plant to prevent an allergic reaction.

A request for additional input data may be provided, and upon receipt of the additional input data, the developmental delay plan can be updated. In some instances, an alert can be provided to the patient, the caregiver, and/or the healthcare provider of the potential developmental delay (e.g., identification of the developmental delay), the developmental delay risk, and the proposed action to reduce the developmental delay risk of the patient. For example, if a health risk tool and associated machine learning models determine the patient is at risk for a developmental delay, this risk, along with the developmental delay response plan can be sent as an alert to the patient, a healthcare provider, a caregiver, or any combination thereof

In some examples, a health risk tool can detect early signs of developmental delays, for instance using machine learning, and provide notifications to a computing device of the patient, health care provider, or caregiver. This detection, for instance, can be determined using the developmental baseline, baseline deviations, patient risk factors, classified and deciphered healthcare provider data, and environmental risks and triggers. A plurality of machine learning models can be used; for instance, a first model can be used for tabular data, a second model for image data, and a third model for audio data, among other models, can be used. The output of the plurality of machine learning models can be used to detect the early signs.

For instance, cameras may gather videos and image sequences of a child attempting to write his or her name. An audio collection device may collect audio of a caregiver yelling at a child while the child is attempting to write his or her name. Pre-trained machine learning models for each data type can identify and classify these actions as “attempting to write name”, “writing name”, “child being yelled at”, etc. Using these classifications, the same or a different machine learning models may determine the combination of these may result in the patient being at risk of developing a writing delay. A wearable device may indicate the child's blood pressure and stress levels are increasing while attempting to write his or her name while being yelled at. A machine learning model may determine a combination of rising blood pressure and stress levels are leading the patient towards a potential developmental delay. Such determinations and detections can be transmitted to a computing device of the patient, a healthcare provider, or caregiver.

FIG. 4 is another functional diagram representing a processing resource 452 in communication with a memory resource 450 having instructions 454, 456, 458, 459, 460, 462 written thereon in accordance with a number of embodiments of the present disclosure. In some examples, the processing resource 452 (herein after referred to as the first processing resource 452) and the memory resource 450 comprise a device and may be analogous to the processing resource 352 and the memory resource 350 illustrated in FIG. 3, and/or health risk tool 108 illustrated in FIG. 1.

The instructions 454, when executed by a processing resource such as the first processing resource 452 can include instructions to receive at the first processing resource 452, the memory resource 450, or both, patient image data, patient audio data, or both, via first signaling configured to monitor the patient. The first signaling may be received from a camera, audio collection device, mobile device, or other source able to capture image data and audio data. For instance, patient image data can include images, videos, and image sequences of a patient's actions, environment, interactions, appearance, etc. The patient audio data can include speech patterns, environmental data (e.g., arguing in background, loud background noise, etc.), conversations, etc. The patient image data and the patient audio data can be received, in some instances, in real time. In some examples, the patient image data and the patient audio data can be received as previously recorded images, videos, image sequences, and sounds.

The instructions 456, when executed by a processing resource such as the first processing resource 452 can include instructions to receive at the first processing resource 452, the memory resource 450, or both, patient health data and patient environmental data via second signaling configured to receive input from the patient, a health care provider, a sensor, or any combination thereof. The second signaling may be received from a health sensor, health monitor, wearable device, mobile device of the patient, or any combination thereof. In some examples, the first signaling may be received in real time. For instance, this first signaling can include real time patient health data such as a heartrate, rashes, blood pressure, or blood sugar level, among others. The first signaling may also include data received from a mobile device of the patient such as manually input data (e.g., via an application) such as age, weight, height, physician information, allergies, etc. In some instance, patient health data may be received from a health care provider (e.g., vitals, bloodwork results, etc.). The patient health data can include health symptoms, a health event (e.g., allergic reaction, surgery, etc.), personal health information of the patient, identifying information of the patient, a location of the patient, data collected by a health monitor, manually input data of the patient, or any combination thereof.

The environmental data can include lighting, screen time, diet, humidity, temperature, sound, caregiver actions, community traits, socioeconomic status, caregiver traits, social interactions, or any combination thereof. The environmental data, for instance, can be collected via manual input and/or using environmental sensors such as temperature or other weather sensors, screen time sensors, food tracking sensors, lighting sensors, etc. In some examples, the health care provider data can include data associated with medical research or treatment databases including common and rare developmental symptoms, treatments, or trends in genders and ages, among other categories (e.g., considering environmental factors.

In some examples, the patient health data, the patient environmental data, the patient image data, the patient audio data, health care provider data, and/or additional environmental data carry different weights within trained machine learning models. For instance, patient health data may be given a great weight than health care provider data, as the patient health data is specific to the patient. The weights can change as more data is received and the machine learning models are updated. For example, if the patient experiences a stutter each time her or she is in a particular environment, all or some environmental factors (and associated images or sounds) may be given a higher weight. The trained machine learning models may carry weights, as well. For instance, different data (e.g., tabular, image, audio, etc.) may be analyzed using different machine learning models appropriate for that data type. A particular type of data, for instance tabular data, may be given a higher weight than audio data, meaning the associated tabular machine learning model may carry a higher weight than a speech recognition machine learning model, for instance.

The instructions 458, when executed by a processing resource such as the first processing resource 452 can include instructions to write from the first processing resource 452 to the memory resource 450 the patient health data and patient environmental data and the patient image data, the patient audio data, or both. In some examples, the memory resource 450 or other storage can include a database including generic developmental delay information including generic developmental delays and associated diagnoses and treatments. The other storage, in some examples, may include cloud storage (e.g., secure cloud storage).

The instructions 459, when executed by a processing resource such as the first processing resource 452 can include instructions to determine, at the first processing resource 452 or a second processing resource, a developmental delay risk of the patient using a plurality of trained machine learning models, input data representative of the written patient health data and patient environmental data and the written patient image data, patient audio data, or both. Put another way, using the plurality of machine learning models, a probability the patient may experience a developmental delay and at what point that may occur is determined. The developmental delay risk can consider several factors associated with the patient to determine a set of circumstances most likely to foreshadow the developmental delay. Deviations from norms of same aged children may indicate a developmental delay risk and are flagged by the machine learning model.

In some examples, a first one of the plurality of machine learning models uses the patient health data and at least a portion of the environmental data to perform multi-class classification. A second one of the plurality of machine learning models can include a convolutional neural network machine learning model to perform multi-class classification on the patient image data, and a third one of the plurality of machine learning models can include a speech recognition machine learning model to perform multi-class classification on the patient audio data. The determinations made using the plurality of machine learning models can be considered together to determine a developmental delay risk, for instance.

The instructions 460, when executed by a processing resource such as the first processing resource 452 can include instructions to identify, at the first processing resource 452 or the second processing resource, output data representative of a developmental delay plan for the patient using the plurality of trained machine learning models, input data representative of the written patient health data and patient environmental data, the written patient image data, patient audio data, or both, and input data representative of the developmental delay risk. The developmental delay plan can include a developmental delay risk and a plan of action for addressing the developmental delay risk. For instance, the developmental delay plan can include how to immediately address the risk, as well as an ongoing plan to address the developmental delay risk and/or potential developmental delay symptoms.

The instructions 462, when executed by a processing resource such as the first processing resource 452 can include instructions to transmit the output data representative of the developmental treatment plan to the patient, a health care provider, or any combination thereof. For instance, a patient may receive an immediate alert if a determination is made that the patient is at a high development delay risk or may receive periodic updates if it is determined the patient is at a low developmental delay risk. For instance, if the patient is experiencing a threshold number of potential symptoms or variations from norm of same aged children, the patient, caregiver, or healthcare provider may receive an audio, physical, or other alert including a developmental delay risk and a developmental delay plan to reduce the risk.

While examples associated with FIGS. 3 and 4 describe developmental delay risks, embodiments are not so limited. For instance, a health risk tool may be utilized to determine a risk of allergic reaction, negative adult behavior, mental health condition, or other health conditions using the plurality of input data received at the health risk tool (e.g., health risk tool 108, processing resources 352, 452, and memory resources 350, 450).

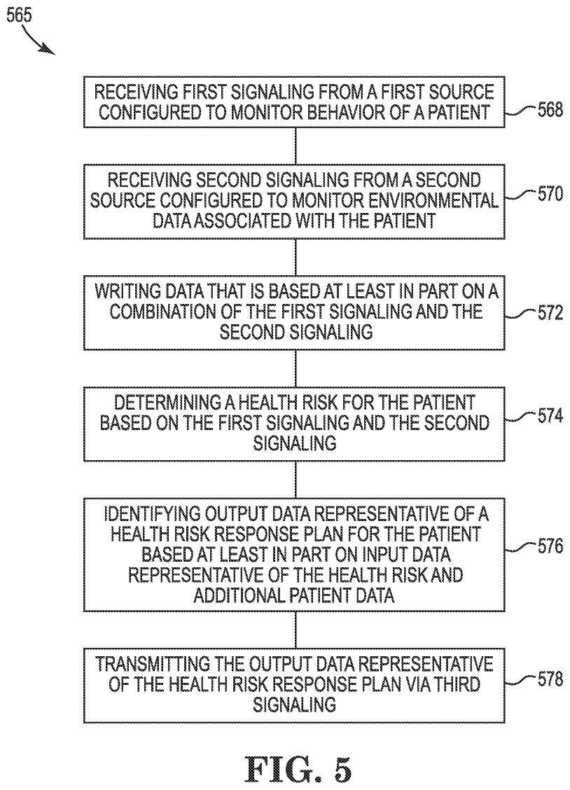

FIG. 5 is a flow diagram representing an example method 565 for health risk determination in accordance with a number of embodiments of the present disclosure. The method 565 may be performed, in some examples, using a health risk tool 108 and/or a device such as those described with respect to FIGS. 2, 3, and 4.

The method 565, at 568, can include receiving at a first processing resource, first signaling from a first source configured to monitor behavior of a patient. The behavior may be monitored in real time, for example. For instance, the first source may include a camera, audio collection device, or other device for collecting image data and audio data of the patient. The image data in some instances can include video data separated into frames such that the first signaling includes image sequences. In some examples, the first source includes a sensor for monitoring patient health data such as oxygen levels, heartrates, body temperature, etc. Patient health data may include, in some instances, data from a health care provider visit (e.g., bloodwork, vital signs, etc.). Patient health data may be received via manual input (e.g., via a mobile device application) in some examples.

At 570, the method 565 can include receiving, at the first processing resource, second signaling from a second source configured to monitor environmental data associated with the patient. The environmental data may be monitored in real time, for example. The second source may include a camera, audio collection device, or other device for collecting image data and audio data of the patient. The image data in some instances can include video data separated into frames such that the second signaling includes image sequences. In some examples, the second source can include a sensor for monitoring environmental data such as temperature data, lighting data, humidity levels, etc. Environmental data may be received via manual input (e.g., via a mobile device application) in some examples.

In some instances, health provider data can be received, for example, including medical research and/or databases of generic health symptoms, triggers, and/or treatment data. For instance, this can include big data compiled by a health care provider or other source for different patients.

At 572, the method 565 can include writing from the first processing resource to a memory resource coupled to the first processing resource data that is based at least in part on a combination of the first signaling and the second signaling. The written data can be saved at the memory resource for use in determination of a current or future health risk response plan, such as a developmental delay plan.

The method 565, at 574, can include determining, at the first processing resource or a different, second processing resource, a health risk for the patient based on the first signaling and the second signaling. For instance, determining the health risk can include determining a likelihood that the patient is at risk of a particular health condition or currently has the particular health condition. The health risk, for instance, may include a developmental delay risk, mental health condition risk, allergy risk, or other health risk.

In some examples, determining the health risk can include utilizing a plurality of trained machine learning models to determine the health risk based on data associated with the first signaling, the second signaling, and previously received signaling and associated data associated with previous health risk response plans. The plurality of trained machine learning models can perform multi-class classification on tabular data, image data, and language data can be utilized. For instance, different machine learning models utilize different data (e.g., tabular, image, audio) to determine different probabilities of a patient's health risk. The plurality of machine learning models can be utilized in congruence to determine the health risk.

For instance, as data is received at the each of the plurality of machine learning models, the health risk and associated health risk response plan is updated. If a previous health risk response plan had elements that worked and elements that did not, the health risk response plan and health risk can be updated as new and updated data are received at the plurality of machine learning models. For instance, the health risk can be updated in response to receiving at the first processing resource additional first signaling, second signaling, or any combination thereof and based at least in part on feedback received at the first processing resource associated with outcomes of the output data representative of the health risk response plan.

For example, a child may be struggling to roll over, but a health risk response plan may indicate that the child is more likely to roll over when a toy is placed nearby. This can be communicated to the caregiver, and a potential developmental delay may be prevented and/or addressed accordingly.

At 576, the method 565 can include identifying, at the first processing resource or the different, second processing resource, output data representative of a health risk response plan for the patient based at least in part on input data representative of the health risk and additional patient data stored in a portion of the memory resource or other storage accessible by the first processing resource. In some examples, identifying the output data representative of the health risk response plan includes utilizing the same or a different plurality of trained machine learning models to identify the output data representative of the health risk response plan based on data associated with the first signaling, the second signaling, the health risk, and previously received signaling and associated data associated with previous health risk response plans, as noted above. The health risk response plan, for instance, can include the health risk and an associated plan to address the risk including, for instance, actions to take to reduce triggers (e.g., avoid allergen, speak slower, etc.). The health risk response plan can be transmitted to the patient, caregiver, healthcare provider, or any combination thereof.

At 578, the method 565 can include transmitting the output data representative of the health risk response plan via third signaling. For instance, identifying the output data representative of the health risk response plan can include identifying an alert to transmit to a computing device of the caregiver, health care provider, and/or patient and identifying a proposed action and associated instructions to reduce the health risk of the patient.

In some examples, the method 565 can include receiving at the first processing resource via an application of the computing device accessible by the patient or a different a mobile device of the patient, manual input from the patient comprising personal patient data, patient health data, environmental data, health care provider data, or a combination thereof and writing from the first processing resource to the memory resource coupled to the first processing resource data that is based at least in part on a combination of the first signaling, the second signaling, and the manual input. Put another way, the patient's health risk and health risk response plan can be monitored and updated via an application. The patient can input additional data (e.g., weight, age, height, odd symptoms, environmental conditions, etc.), and a health care provider can input additional data (e.g., new research, test results, etc.). This additional data can be used by the plurality of machine learning models to determine a health risk and health risk response plan.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and processes are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A method, comprising:
receiving, at a first processing resource, first signaling from a first source configured to monitor behavior of a patient;
receiving, at the first processing resource, second signaling from a second source configured to monitor environmental data associated with the patient;
writing from the first processing resource to a memory resource coupled to the first processing resource data that is based at least in part on a combination of the first signaling and the second signaling;
determining, at the first processing resource or a different, second processing resource, a health risk for the patient based on the first signaling and the second signaling;
identifying, at the first processing resource or the different, second processing resource, output data representative of a health risk response plan for the patient based at least in part on input data representative of the health risk and additional patient data stored in a portion of the memory resource or other storage accessible by the first processing resource; and
transmitting the output data representative of the health risk response plan via third signaling.

2. The method of claim 1, wherein identifying the output data representative of the health risk response plan comprises utilizing a plurality of trained machine learning models to identify the output data representative of the health risk response plan based on data associated with the first signaling, the second signaling, the health risk, and previously received signaling and associated data associated with previous health risk response plans.

3. The method of claim 1, wherein determining the health risk comprises utilizing a plurality of trained machine learning models to perform multi-class classification on tabular data, image data, and language data.

4. The method of claim 1, wherein determining the health risk comprises determining a likelihood that the patient is at risk of a particular health condition or currently has the particular health condition.

5. The method of claim 1, wherein identifying the output data representative of the health risks response plan comprises:
identifying an alert to transmit to a computing device of the patient; and
identifying a proposed action and associated instructions to address the health risk of the patient.

6. The method of claim 1, further comprising updating the health risk in response to receiving at the first processing resource additional first signaling, second signaling, or any combination thereof and based at least in part on feedback received at the first processing resource associated with outcomes of the output data representative of the health risk response plan.

7. The method of claim 1, further comprising:
receiving at the first processing resource via an application of a computing device accessible by the patient or a different mobile device of the patient, manual input from the patient comprising personal patient data, patient health data, environmental data, health care provider data, or a combination thereof; and
writing from the first processing resource to the memory resource coupled to the first processing resource data that is based at least in part on a combination of the first signaling, the second signaling, and the manual input.

8. The method of claim 1, wherein determining the health risk comprises determining a developmental delay risk.

9. The method of claim 1, wherein the first signaling, the second signaling, or both, comprise image sequences.

10. A non-transitory machine-readable medium comprising a processing resource in communication with a memory resource having instructions executable to:
receive at a processing resource, the memory resource, or both, a plurality of input data from a plurality of sources, the plurality of sources comprising at least two of: a mobile device of a patient, a medical device, a portion of the memory resource or other storage, manually received input, and environmental sensors;
write from the processing resource to the memory resource the received plurality of input data;
identify, using a plurality of machine learning models, at the processing resource or a different processing resource, output data representative of a developmental delay plan including a proposed action to identify the developmental delay, address the developmental delay, or both, based at least in part on input data representative of the data written from the processing resource; and
transmit the output data representative of the developmental delay plan to the patient, a caregiver, a health care provider, or any combination thereof.

11. The medium of claim 10, further comprising the instructions executable to identify the output data representative of the developmental delay plan based at least in part on generic developmental patient information and generic developmental delay treatment information stored in a portion of the memory resource or other storage accessible by the processing resource.

12. The medium of claim 10, further comprising the instructions executable to identify the output data representative of the developmental delay plan based at least in part on patient medical history information stored in a portion of the memory resource or other storage accessible by the processing resource.

13. The medium of claim 10, wherein the plurality of input data comprises patient health data, environmental data, or any combination thereof.

14. The medium of claim 10, wherein the plurality of machine learning models comprises a speech recognition model to perform multi-class classification, a convolutional neural network model to perform multi-class classification, and a machine learning model using tabular data to perform multi-class classification.

15. A non-transitory machine-readable medium comprising a first processing resource in communication with a memory resource having instructions executable to:
receive at the first processing resource, the memory resource, or both, patient image data, patient audio data, or both, via first signaling configured to monitor the patient;
receive at the first processing resource, the memory resource, or both, patient health data and patient environmental data via second signaling configured to receive input from the patient, a health care provider, a sensor, or any combination thereof;
write from the first processing resource to the memory resource the patient health data and patient environmental data and the patient image data, the patient audio data, or both;
determine, at the first processing resource or a second processing resource, a developmental delay risk of the patient using a plurality of trained machine learning models, input data representative of the written patient health data and patient environmental data and the written patient image data, patient audio data, or both;
identify, at the first processing resource or the second processing resource, output data representative of a developmental delay plan for the patient using the plurality of trained machine learning models, input data representative of the written patient health data and patient environmental data, the written patient image data, patient audio data, or both, and input data representative of the developmental delay risk; and
transmit the output data representative of the developmental delay treatment plan to the patient, a health care provider, a caregiver, or any combination thereof.

16. The medium of claim 15, wherein the patient health data, the patient environmental data, the patient image data, and the patient audio data carry different weights within the plurality of trained machine learning models.

17. The medium of claim 15, wherein:
a first one of the plurality of machine learning models uses the patient health data and at least a portion of the environmental data to perform multi-class classification;
a second one of the plurality of machine learning models comprises a convolutional neural network machine learning model to perform multi-class classification on the patient image data; and
a third one of the plurality of machine learning models comprises a speech recognition machine learning model to perform multi-class classification on the patient audio data.

18. The medium of claim 15, wherein the instructions executable to receive the patient image data, patient audio data, or both via first signaling configured to monitor patient health data comprise instructions executable to receive the patient image data, patient audio data, or both via signaling from at least one of a health sensor, health monitor, wearable device, camera, audio collection device, or mobile device of the patient.

19. The medium of claim 15, wherein the patient health data, the patient environmental data, the patient image data, the patient audio data, or any combination thereof is received in real time.

20. The medium of claim 15, wherein the patient environmental data comprises lighting, screen time, diet, humidity, temperature, sound, caregiver actions, community traits, socioeconomic status, caregiver traits, social interactions, or any combination thereof.
